(19) United States
(12) Patent Application Publication
(10) Pub. No.: US 2021/0048439 A1
(43) Pub. Date: Feb. 18, 2021

(54) CAGED AMINO ACIDS FOR CONTROLLED METABOLIC INCORPORATION AND METHODS OF USE

(71) Applicant: University of Washington, Seattle, WA (US)

(72) Inventors: Cole A. DeForest, Seattle, WA (US); Steven Adelmund, Seattle, WA (US)

(73) Assignee: University of Washington, Seattle, WA (US)

(21) Appl. No.: 16/965,575

(22) PCT Filed: Feb. 1, 2019
(86) PCT No.: PCT/US2019/016288

§ 371 Date: Jul. 28, 2020

Related U.S. Application Data
(60) Provisional application No. 62/625,871, filed on Feb. 2, 2018.

(51) Int. Cl. G01N 33/60; G01N 33/68

(52) U.S. Cl. CPC G01N 33/60; G01N 33/6806

(57) ABSTRACT

The present disclosure features non-canonical or heavy isotope-containing amino acids, where the alpha-amino terminus and/or carboxylic acid terminus is modified with molecular cages. The molecular cage-modified amino acids are precluded from metabolic incorporation into proteins within living bacterial, plant, or mammalian cells, or from cell-free protein expression. Once uncaged, the amino acids are readily recognized by native and/or engineered tRNA synthetases, and can subsequently be incorporated into newly-synthesized proteins during protein translation.

### CROSS-REFERENCE TO RELATED APPLICATION

This application claims the benefit of U.S. Patent Application No. 62/625,871, filed Feb. 2, 2018, the disclosure of which is hereby incorporated by reference in its entirety.

### STATEMENT OF GOVERNMENT LICENSE RIGHTS

This invention was made with Government support under NNX15AJ98H awarded by the NASA Space Grant Consortium. The Government has certain rights in the invention.

### BACKGROUND

Biological processes are staggeringly dynamic and heterogeneous. Although all cells within an organism share a common genome, differential expression of genes into proteins regulate developmental processes, tissue morphogenesis and function, and disease susceptibility and response; and a diverse array of signaling events are governed by a wide variety of intra- and extra-cellular cues. While each protein is encoded by a gene, protein quantity and activity cannot be determined through genomic or transcriptomic analysis; such techniques are blind to post-transcriptional phenomena (e.g., translational regulation, modification, protein-biomolecular interactions), necessitating strategies to directly measure protein identity and abundance.

Recent advances in mass spectrometry and genomic sequencing have enabled high-throughput proteomic analysis, whereby the abundance, turnover, modification, and interactions of thousands of proteins can be measured in minutes. From this has emerged a growing appreciation of the exceptionally dynamic nature of the proteome, which undergoes large-scale biochemical shifts during cellular proliferation, migration, and differentiation. Efforts to quantify temporal variations of the proteome have focused on the pulsed labeling of cultures with specialty amino acids that can distinguish newly synthesized from pre-existing cellular proteins.

One particularly powerful method for interrogating proteomic fluctuations in a cell culture is known as bioorthogonal non-canonical amino acid tagging (BONCAT). In BONCAT, pulsing cells with non-canonical amino acid (ncAA) analogs yields newly synthesized proteins that bear bioorthogonal reactive groups (e.g., azides, alkynes). Metabolically-labeled proteins can be covalently modified with an affinity tag that is then exploited for purification, thereby enabling isolation of proteins synthesized over a short window of time and providing temporally resolved proteomics.

“Heavy” isotopes of standard amino acids, as well as non-canonical amino acids, are utilized (e.g., SILAC: Stable Isotope Labeling by Amino acids in Cell culture; BONCAT) to identify newly-synthesized proteins in response to environmental stimuli. Such compounds have defined the field of temporally-resolved proteomics, providing researchers with quantitative information about dynamic changes to the proteome in a simple and reliable manner. Existing products to metabolically label the proteome have been recently commercialized and are available through a variety of prominent chemical suppliers including Thermo Fisher, Invitrogen, and Click Chemistry Tools. However, despite the utility of these strategies in determining when a protein was synthesized, they are unable to provide information about where it was created. Such spatial information is vital for fundamental biological studies as well as in determining novel therapeutic targets for disease treatment.

There is presently a need for spatiotemporally-resolved proteomics, where information regarding the location and time of a protein's synthesis can be simultaneously obtained. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a method of analyzing a protein composition in a living cell or organism, including:

providing a caged non-canonical amino acid, a caged heavy isotope-labeled amino acid, or a combination thereof, to a living cell or organism, wherein the caged amino acid is configured to be uncaged when exposed to a stimulus;

providing a stimulus to uncage the caged non-canonical amino acid or caged heavy isotope-labeled amino acid;

growing the living cell or organism to incorporate the uncaged amino acid in a growing protein; and

analyzing the living cell or organism for incorporation of the non-canonical amino acid, the heavy isotope-labeled amino acid, or both, into a protein.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a scheme showing light-activated bioorthogonal non-canonical amino acid tagging (laBONCAT). Photocaged non-canonical amino acids (ncAA) become available for stochastic incorporation into newly translated proteins following directed light exposure. Bioorthogonal handles installed by ncAAs can be exploited for protein purification prior to quantitative proteomics or fluorescent tagging for visualization.

FIG. 2A is a scheme showing the chemical structures of L-methionine (Met), L-azidohomoalanine (Aha), and photocaged Aha (NPPOC-Aha).

FIG. 2B is a scheme showing SPAAC, a biorthogonal reaction for labeling azides incorporated into proteins with strained cyclooctynes.

FIG. 2C is a scheme showing photolysis of NPPOC-Aha upon light exposure, yielding free Aha for incorporation into newly synthesized proteins.

FIG. 2D is a graph showing the kinetic analysis of NPPOC-Aha photolysis, demonstrating rapid uncaging suitable for biological sampling.

FIG. 2E is a graph showing the concentration of a carboxyfluorescein-labeled bicyclononyne (FAM-BCN) as a function of time, where the carboxyfluorescein-labeled bicyclononyne provides a fluorescent tag for labeling azide-functionalized proteins.

FIG. 3A is a photograph showing the analysis of fluorescently labeled protein lysate by SDS-PAGE. In vitro incorporation of free L-azidohomoalanine (Aha) and Aha after photoliberation provides azide-functionalized proteins for fluorescent tagging by carboxyfluorescein-labeled bicyclononyne (FAM-BCN). Only samples incubated with free Aha or light-treated photocaged Aha (NPPOC-Aha) are fluorescently labeled. Coomassie staining indicates near-uniform protein loading.

FIG. 3B is a graph showing the effect of light intensity and NPPOC-Aha concentration on azide incorporation into newly synthesized proteins, based on protein fluorescence.

FIG. 3C is a graph showing the degree of incorporation from FIG. 3B, normalized for expected free Aha concentration.

FIG. 3D is a graph showing that light irradiation itself does not impact ncAA incorporation of Aha (*p<0.05 by one-way ANOVA followed by Tukey's test).

FIG. 3E is a graph showing that a significant amount of NPPOC-Aha remains stable over several hours in media and in contact with live cells suitable for labeling new proteins in tissue culture.

FIG. 3F is a photograph showing that, following photomediated Aha incorporation, newly synthesized proteins can be isolated by affinity purification for downstream proteomic analysis. Fractions from left to right: flow through 1 (F1), wash 1-5 (W1-5), and elution 1 (E1).

FIG. 4A is a photograph showing that irradiation of photocaged L-azidohomoalanine (NPPOCAha) yields free Aha for ncAA in vitro incorporation. Fluorescent tagging of fixed cells demonstrates similar fluorescent labeling of Aha and irradiated NPPOC-Aha, but not L-methionine (Met) and unexposed NPPOC-Aha (scale bar=100 μM).

FIG. 4B is a photograph showing that metabolic labeling of cells in synthetic tissue is confined to regions near user-defined exposure (hashed area). Actin staining (bottom) remains uniform across sample (scale bar=1 mm).

### DETAILED DESCRIPTION

The present disclosure features non-canonical or heavy isotope-containing amino acids, where the alpha-amino terminus and/or carboxyl acid terminus of the amino acid is modified with molecular cages. The molecular cage-modified amino acids are precluded from metabolic incorporation into proteins within living bacterial, plant, or mammalian cells, or from cell-free protein expression. Once uncaged, the amino acids are readily recognized by native and/or engineered tRNA synthetases, and can subsequently be incorporated into newly-synthesized proteins during protein translation. The caging strategy can be applied to any amino acid, but is of particular interest for “heavy” isotopes of standard amino acids or non-canonical amino acids whose mass fingerprint and expanded chemical functionality can be exploited for quantitative proteomic studies. As an example, by caging non-canonical amino acids with moieties that can be readily removed with a stimulus, such as light, this disclosure provides the first tools to enable spatiotemporally-resolved proteomics, permitting investigation of proteomic response to stimuli at desired times and locations within a given cell culture.

As used herein, molecular “cages” refer to protective moieties that alter chemical functionality while present, but can be readily removed upon exposure to a specific stimulus (i.e., light, enzyme, small molecules, nucleic acids, temperature, pH, ultrasound, and/or mechanical force). If the cage masks an important biochemical feature, its removal can restore the implicated biological function. For example, molecular cages can be used to mask amino acid side chains of bioactive peptides and proteins. The state of these molecules (caged or uncaged) can be controlled and used to investigate molecular processes or physiological phenomena.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Furthermore, references have been made to patents and printed publications throughout this specification. Each of the above-cited references and printed publications are individually incorporated herein by reference in their entirety.

As used herein, the term “non-canonical amino acid” refers to amino acids that are other than the 20 natural proteinogenic amino acids that are encoded directly by the codons of the universal genetic code.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)C₁-C₆ alkyl indicate the same functionality; similarly arylalkyl and -alkylaryl indicate the same functionality.

As used herein, the term “alkyl” refers to a straight or branched chain hydrocarbon containing from 1 to 10 carbon atoms, unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkylene” refers to a linking alkyl group. The linking alkyl group can be a straight or branched chain; examples include, but are not limited to —CH₂—, —CH₂CH₂—, —CH₂CH₂CHC(CH₃)—, and —CH₂CH(CH₂CH₃)CH₂—.

As used herein, the term “alkenyl” refers to a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

As used herein, the term “alkenylene” refers to a linking alkenyl group.

As used herein, the term “alkynyl” refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, “alkynylene” refers to a linking alkynyl group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, the term “arylene” refers to a linking aryl group.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “cycloalkylene” refers to a linking cycloalkyl group.

As used herein, “heteroalkyl” refers to an alkyl group having at least one heteroatom such as sulfur, oxygen, or nitrogen.

As used herein, “heteroalkylene” refers to a linking heteroalkyl group.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “heterocycloalkyl” refers to non-aromatic heterocycles including cyclized alkyl, alkenyl, and alkynyl groups where one or more of the ring-forming carbon atoms are replaced by a heteroatom such as an O, N, or S atom. Heterocycloalkyl groups can be mono- or polycyclic (e.g., having 2, 3, 4 or more fused rings or having a 2-ring, 3-ring, 4-ring spiro system (e.g., having 8 to 20 ring-forming atoms). Heterocycloalkyl groups include monocyclic and polycyclic groups. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-I,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “alkoxy” refers to an —O-alkyl group. Example alkoxy groups include methoxy, ethoxy, propoxy (e.g., n-propoxy and isopropoxy), t-butoxy, and the like.

As used herein, “cycloalkoxy” refers to an —O-cycloalkyl group.

As used herein, “heterocycloalkoxy” refers to an —O-heterocycloalkyl group.

As used herein, “aryloxy” refers to an —O-aryl group. Example aryloxy groups include phenyl-O—, substituted phenyl-O—, and the like.

As used herein, “heteroaryloxy” refers to an —O-heteroaryl group.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “halo” or “halogen” includes fluoro, chloro, bromo, and iodo.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkenyl” refers to an alkenyl group having one or more halogen substituents.

As used herein, “haloalkynyl” refers to an alkynyl group having one or more halogen substituents.

As used herein, “haloalkoxy” refers to an —O-(haloalkyl) group.

As used herein, “amino” refers to NH₂.

As used herein, “alkylamino” refers to an amino group substituted by an alkyl group.

As used herein, “dialkylamino” refers to an amino group substituted by two alkyl groups that can be the same, or different from one another.

As used herein, “ether” refers to a group comprising an oxygen atom connected to two alkyl or aryl groups. As used herein, a “vinyl ether” refers to an ether comprising a carbon-carbon double bond bound to the oxygen atom.

As used herein, an “electron donating substituent” refers to a substituent that adds electron density to an adjacent pi-system, making the pi-system more nucleophilic. In some embodiments, an electron donating substituent has lone pair electrons on the atom adjacent to pi-system. In some embodiments, electron donating substituents have pi-electrons, which can donate electron density to the adjacent pi-system via hyperconjugation. Examples of electron donating substituents include O—, NR₂, NH₂, OH, OR, NHC(O)R, OC(O)R, aryl, and vinyl substituents.

As used herein, “unsaturated bond” refers to a carbon-carbon double bond or a carbon-carbon triple bond.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Caged Amino Acids

The present disclosure features a method of analyzing a protein composition in a living cell or organism, including:

1) providing a caged non-canonical amino acid, a caged heavy isotope-labeled amino acid, or a combination thereof, to a living cell or organism, wherein the caged amino acid is configured to be uncaged when exposed to a stimulus;

2) providing a stimulus to uncage the caged non-canonical amino acid or caged heavy isotope-labeled amino acid;

3) growing the living cell or organism to incorporate the uncaged amino acid in a growing protein; and

4) analyzing the living cell or organism for incorporation of the non-canonical amino acid, the heavy isotope-labeled amino acid, or both, into a protein.

In one embodiment, the present disclosure provides a method for preventing, limiting, reducing, inhibiting, or controlling metabolic incorporation into proteins within living bacterial, plant, or mammalian cells, including providing a composition including a caged amino acid with the cells, exposing the caged amino acid to a stimulus to selectively uncage the caged amino acid, and allowing the cell to grow in culture or in a tissue. The caged amino acid can be a canonical, non-canonical, fixed isotope, or mixed isotope amino acid. In a preferred embodiment, the caged amino acid is a caged non-canonical amino acid or caged heavy isotope-labeled amino acid.

In some embodiments, the living cell or organism includes a bacterial cell, a plant cell, a mammalian cell, or a tissue sample (e.g., a tissue slice).

The non-canonical amino acid and the heavy isotope-labeled amino acid are caged at the alpha-amino acid terminus and/or the carboxylic acid terminus of the amino acid, but not at the side chain. The caged non-canonical amino acid and the caged heavy isotope-containing amino acid are inactive in protein synthesis. Once uncaged on at least one of the alpha-amino acid terminus and/or the carboxylic acid terminus on the amino acid (upon exposure to a stimulus to which the caging group is sensitive to), the uncaged non-canonical amino acid or uncaged heavy isotope-labeled amino acid can be incorporated into backbone of the growing protein. Thus, the uncaged non-canonical amino acids or heavy isotope-labeled amino acid reacts at the alpha-amino acid terminus and/or at the carboxylic acid terminus, but not at the side chains, to form amide bonds on the growing protein.

The stimulus for uncaging the caged amino acid can be selected from light having a predetermined wavelength, an enzyme, exposure to a small molecule, exposure to a nucleic acid, a predetermined temperature, a predetermined pH, ultrasound, a reductant, an oxidant, a predetermined mechanical force, and any combination thereof. In some embodiments, the stimulus is light of a certain wavelength. In certain embodiments, the stimulus is ultrasound. In some embodiments, the stimulus is an enzyme, a predetermined temperature, and/or a predetermined pH. The stimulus and uncaging process can be mild and cytocompatible. Without wishing to be bound by theory, it is believed that a desirable uncaging reaction has rapid uncaging kinetics, such that the liberation of uncaged amino acids is not rate-limiting compared with the biological processes under study.

To obtain spatiotemporal proteomic information from the living cell or the living organism, analyzing the living cell or organism for incorporation of the non-canonical amino acid and/or the heavy isotope-labeled amino acid into a protein can include obtaining a cell population (e.g., at any given time after uncaging the caged non-canonical amino acid and/or the heavy isotope-labeled amino acid), lysing the cells, performing mass spectrometry on proteins obtained from cell lysis, and identifying the proteins containing the non-canonical amino acid, the heavy isotope-labeled amino acid, or both. In some embodiments, instead or in addition to performing mass spectrometry, the incorporated non-canonical and/or heavy isotope labeled amino acid can be detected by fluorescence or by affinity purification, and the proteins containing the non-canonical and/or heavy isotope labeled amino acid can be identified by the detection of a fluorescent tag or detection of a protein binding event.

The analysis can further include obtaining the time at which the non-canonical amino acid is incorporated into the protein in the living cell or organism (e.g., depending on when the non-canonical amino acid is uncaged by application of a stimulus, and thus becoming available for incorporation into the protein), and/or obtaining the location at which the protein is synthesized in the living cell or organism. In some embodiments, analyzing the living cell or organism includes obtaining both the time and location at which the protein is synthesized in the living cell or organism.

In some embodiments, rather than administering a single kind of caged non-canonical amino acid or heavy isotope labeled amino acid, a plurality of caged non-canonical amino acids, a plurality of caged heavy isotope-labeled amino acids, or a combination thereof, is provided to a living cell or organism, and each caged amino acid is configured to be uncaged when exposed to one or more stimuli.

For example, the caged amino acid can be caged on the carboxylic acid terminus. In some embodiments, the caged amino acid has a moiety having the structure of —C(O)-(caging group), where the —C(O)— is derived from the carbonyl group in the carboxylic acid terminus of the amino acid, and the caging group is selected from an alkoxy, an alkoxy substituted with C₁-C₆ alkylcarbonyloxy or substituted with cycloalkylcarbonyloxy, a heterocycloalkoxy, an arylalkoxy optionally substituted with 1, 2, or 3 substituents independently selected from nitro and alkoxy, and a thioalkyl moiety; or the caged amino acid has a heterocycloalkyl caging moiety where at least 2 ring-forming atoms in the heterocyloalkyl caging moiety (e.g., —C(O)—) are derived from the carboxylic acid terminus of the amino acid (e.g., a dihydrooxazolyl moiety).

In some embodiments, the caged amino acid has a moiety having the structure of —C(O)-(caging group), where the —C(O)— is derived from the carboxylic acid terminus of the amino acid, and the caging group is selected from an enzyme-cleavable alkoxy optionally substituted with C₁-C₆ alkylcarbonyloxy or cycloalkylcarbonyloxy, an ultrasound cleavable heterocycloalkoxy, a light-cleavable arylalkoxy optionally substituted with 1, 2, or 3 substituents independently selected from nitro and alkoxy, and a pH (e.g., acid) cleavable thioalkyl; or the caged amino acid has an oxidation or reduction cleavable heterocycloalkyl caging moiety on the carboxylic acid terminus of the amino acid, where at least 2 ring-forming atoms in the heterocyloalkyl caging moiety (e.g., —C(O)—) are derived from the carboxylic acid terminus of the amino acid (e.g., an oxidation or reduction cleavable dihydrooxazolyl moiety).

For example, the caged amino acid can be caged on the carboxylic acid terminus. In some embodiments, the caged amino acid has a moiety having the structure of —C(O)-(caging group), where the —C(O)— is derived from the carboxylic acid terminus of the amino acid, and the caging group is selected from:

an alkoxy

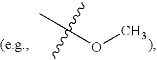 [SMILES: *OC]

an alkoxy substituted with C₁-C₆ alkylcarbonyloxy or cycloalkylcarbonyloxy (e.g.,

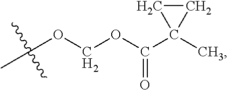 [SMILES: *OCOC(=O)C1(C)CC1]

enzyme cleavable),

a heterocycloalkoxy (e.g.,

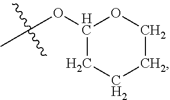 [SMILES: *OC1CCCCO1]

ultrasound cleavable),

an arylalkoxy, wherein the arylalkoxy is optionally substituted with 1, 2, or 3 substituents independently selected from nitro and alkoxy (e.g.,

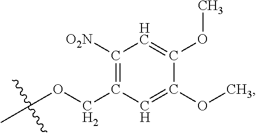 [SMILES: *OCc1cc(OC)c(OC)cc1[N+](=O)[O-]]

light-cleavable), and

a thioalkyl moiety (e.g.,

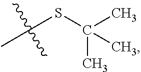 [SMILES: *SC(C)(C)C]

pH cleavable, forming —C(O)S-alkyl thioester, where the —C(O)— is derived from the carboxylic acid terminus of the amino acid),

or the caged amino acid has a heterocycloalkyl caging moiety (e.g.,

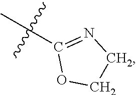 [SMILES: *C1=NCCO1]

where the —C—O— in the heterocycloalkyl group is derived from the carbonyl group on the carboxylic acid terminus of the amino acid, oxidant/reductant cleavable).

In some embodiments, the caged amino acid is caged on the amino terminus and has a moiety having a structure of —NH-(caging group) or —N=(caging group), where the —NH— or —N═ is derived from the amino terminus of the amino acid, and the caging group is selected from an aryl-CH═, heteroaryl-CH═, aryl, arylalkyl, arylcarbonyloxy optionally substituted with 1, 2, or 3 nitro, and arylalkylcarbonyloxy optionally substituted with 1, 2, or 3 nitro.

In some embodiments, the caged amino acid is caged on the amino terminus and has a moiety having a structure of —NH-(caging group) or —N=(caging group), where the —NH— or —N═ is derived from the amino terminus of the amino acid, and the caging group is selected from a pH-cleavable aryl-CH═, a pH-cleavable heteroaryl-CH═, an oxidant or reductant-cleavable aryl, an oxidant or reductant-cleavable arylalkyl, a light-cleavable arylcarbonyloxy optionally substituted with 1, 2, or 3 nitro, and a light-cleavable arylalkylcarbonyloxy optionally substituted with 1, 2, or 3 nitro.

In some embodiments, the caged amino acid is caged on the amino terminus and has a moiety having a structure of —NH-(caging group) or —N=(caging group), where the —NH— or —N═ is derived from the amino terminus of the amino acid, and the caging group is selected from:

an aryl-CH═ or heteroaryl-CH═ (e.g.,

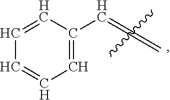 [SMILES: *=Cc1ccccc1]

pH-cleavable, on the amino terminus of the amino acid, forming aryl-CH═N—, where the ═N— is derived from the amino terminus of the amino acid),

an aryl,

an arylalkyl (e.g.,

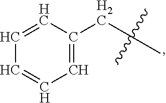 [SMILES: *Cc1ccccc1]

oxidant/reductant cleavable), and

an arylcarbonyloxy and an arylalkylcarbonyloxy, each optionally substituted with 1, 2, or 3 nitro (e.g.,

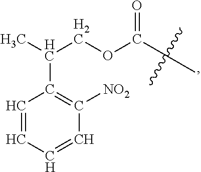 [SMILES: *C(=O)OCC(C)c1ccccc1[N+](=O)[O-]]

light-cleavable, forming a carbamate linkage to the amino terminus of the amino acid).

In some embodiments, the caged amino acid has long-term stability (e.g., hours, days). For example, a caged amino acid that is caged on the amino terminus and having a structure of —NH-(caging group) or —N=(caging group) can be stable for a longer period than caged amino acid that is caged on the carboxylic acid terminus with an ester moiety. The stability can be useful in sampling biological systems, as it decouples media swaps from proteome labeling, and can allow for standardization of experimental conditions.

In some embodiments, the non-canonical amino acid is L-azidohomoalanine (or α-aminobutyric acid), or D- or L-propargylglycine

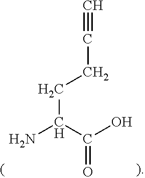

In some embodiments, the heavy isotope-labeled amino acid is a ¹³C-labeled amino acid, such as a ¹³C-labeled arginine and a ¹³C-labeled lysine, or deuterium-labeled amino acids, such as a deuterated leucine.

In some embodiments, the caged non-canonical amino acid, the caged heavy isotope-labeled amino acid, or both, further include a reactive group different from the caging group, such as an azide or an alkyne. The reactive group can be located on a side chain of the amino acid. When incorporated into a protein, the reactive group-containing caged amino acid is further reacted with a labeling molecule having a complementary reactive group. The labeling molecule can be, for example, a fluorescent molecule, a radioactive molecule, a metal, heavy isotope, and/or an affinity tag (e.g., biotin, FLAG tag, polyhistidine tag, albumin-binding protein, maltose-binding protein, bacteriophage tags, calmodulin binding peptide, HaloTag, polyphenylalanine tag, Strep-tag, and/or SNAP-tag). The complementary reactive group can be, for example, azide, alkyne, phosphine-activated moiety, and/or thiol, which can react in strain-promoted azide-alkyne cycloaddition (SPAAC), copper-catalyzed azide-alkyne cycloaddition (CuAAC), Staudinger ligation, and thiol-yne reactions, respectively. In some embodiments, when the synthesized protein is labeled with an affinity tag, the protein can be subjected to affinity purification.

In some embodiments, the caged amino acid is selected from:

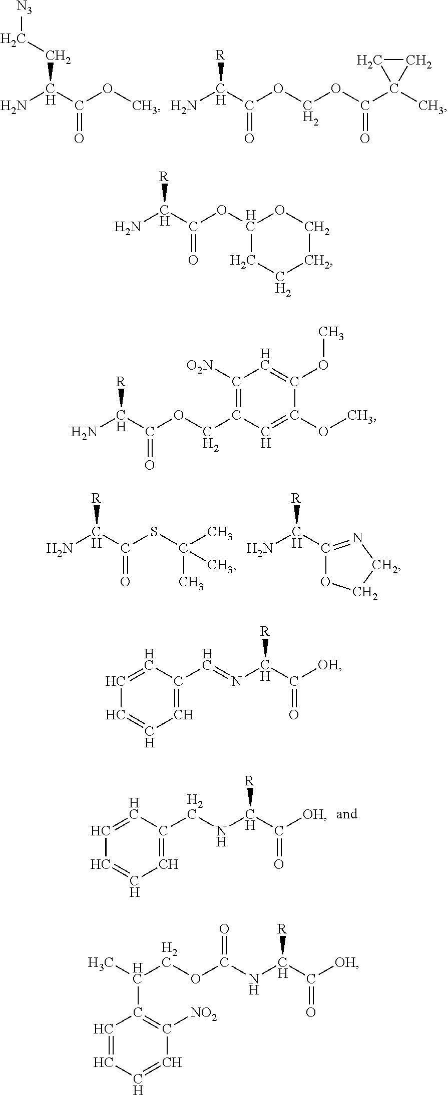

wherein the amino acid is non-canonical or heavy isotope-labeled (e.g., having at least one of H, C, or N replaced by deuterium, ¹³C, or ¹⁵N, respectively); and R is a side chain on the amino acid, optionally substituted with 1, 2, or 3 substituents independently selected from azide and alkyne.

In some embodiments, the caged amino acid is caged at the amino terminus with an activated ester of 2,5-dioxopyrrolidin-1-yl (2-(2-nitrophenyl)propyl) carbonate (NPPOC).

In some embodiments, the methods of the present disclosure can be combined with strategies for pulsed stable isotope labeling by amino acids in cell culture (pSILAC) to purify, identify, and quantify proteins expressed at user-defined regions in culture. For example, both photosensitive heavy isotope-labeled amino acid and photosensitive caged non-canonical amino acid can be added to a cell/tissue culture. The amino acids can be irradiated and activated. The cell/tissue culture can be incubated for a period of time, then the cells can be lysed. The proteins of interest can be extracted by coupling to affinity probe (e.g., biotin) and subjected to affinity column chromatography. The heavy isotopes can be compared in MS/MS to compare proteins of interest under different stimuli (from different treatments/cultures).

The method of the present disclosure can be used in the investigation of heterogeneous protein-related diseases, such as Alzheimer's.

### Methods of Synthesis

The compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

The compounds of this disclosure can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like) HPLC, or preparative thin layer chromatography; distillation; sublimation, trituration, or recrystallization.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene's Protective Groups in Organic Synthesis, 4^{th} Ed., John Wiley & Sons: New York, 2006, which is incorporated herein by reference in its entirety.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE), tetrahydrofuran (THF), dioxane, etc.), esters (such as ethyl acetate, butyl acetate, etc.), halogenated solvents (such as dichloromethane (DCM), chloroform, dichloroethane, tetrachloroethane), dimethylformamide (DMF), dimethylsulfoxide (DMSO), acetone, acetonitrile (ACN), hexamethylphosphoramide (HMPA) and N-methylpyrrolidone (NMP). Such solvents can be used in either their wet or anhydrous forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The compounds of the disclosure can be prepared, for example, using the reaction pathways and techniques as described below.

For example, to form an amine terminus-caged amino acid, an amino acid can be protected at the carboxylic acid terminus, then alkylated with an alkyl halide, or reacted with a carbonyl-containing group to form an imine, or acylated (e.g., with an activated carboxylic acid, such as an acyl chloride) to form an amide; the protected carboxylic acid terminus can then be deprotected. As another example, to form a carboxylic acid terminus-caged amino acid, an amino acid terminus can be protected, then the amino acid can be reacted at the carboxylic acid end by first activating the carboxylic acid (e.g., by forming a N-hydroxysuccinimidyl ester), then reacting with an alcohol or a thiol to form an ester or a thioester; the protected amino acid terminus can then be deprotected.

The following Example describes caged amino acids for controlled metabolic incorporation into proteins.

### Examples

The present Example describes the use of light-activated bioorthogonal non-canonical amino acid tagging (laBONCAT) as a method to selectively label, isolate, and identify newly synthesized proteins at user-defined regions in tissue culture. By photocaging L-azidohomoalanine (Aha), metabolic incorporation into proteins is prevented. The caged compound remains stable for many hours in culture, but can be photochemically liberated rapidly and on demand with spatial control. Upon directed light exposure, the uncaged amino acid is available for local translation, enabling downstream proteomic interrogation via bioorthogonal conjugation. Exploiting the reactive azide moiety present on Aha's amino acid side chain, newly synthesized proteins can be purified for quantitative proteomics or visualized in synthetic tissues with a new level of spatiotemporal control. Shedding light on when and where proteins are translated within living samples, laBONCAT can aid in understanding the progression of complex protein-related disorders.

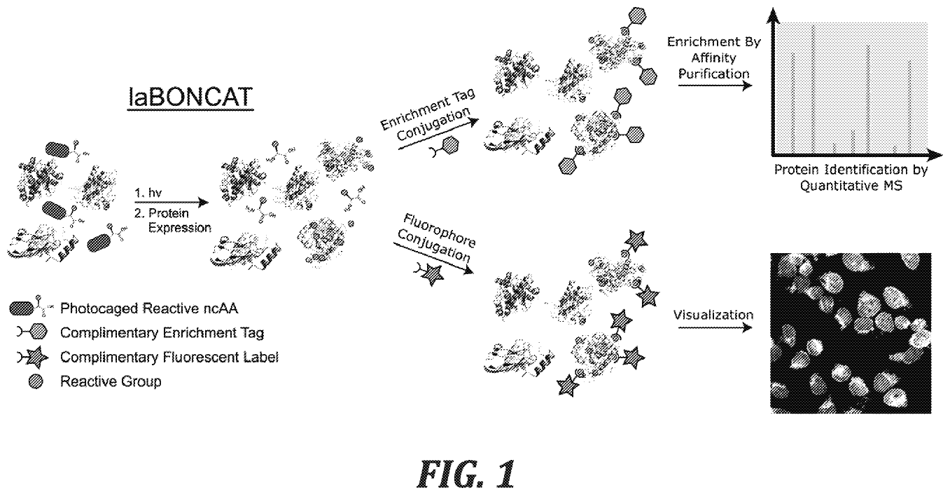

As protein expression, degradation, translocation, and post-translational modification occur at different rates depending on cellular and subcellular location within tissues, BONCAT was controlled within user-defined regions of culture. Recognizing light's unique ability to initiate chemical reactions at a time and place of interest, a light-activated bioorthogonal non-canonical amino acid tagging (laBONCAT) approach (FIG. 1) was developed. This strategy relies on the introduction of a molecular photocage onto the α-amine of a non-canonical amino acid (ncAA), preventing metabolic incorporation into proteins. Upon user-directed light exposure, the ncAA is liberated and made available for incorporation into newly translated proteins. Subsequent labeling and enrichment of these proteins is exploited for proteomic analysis.

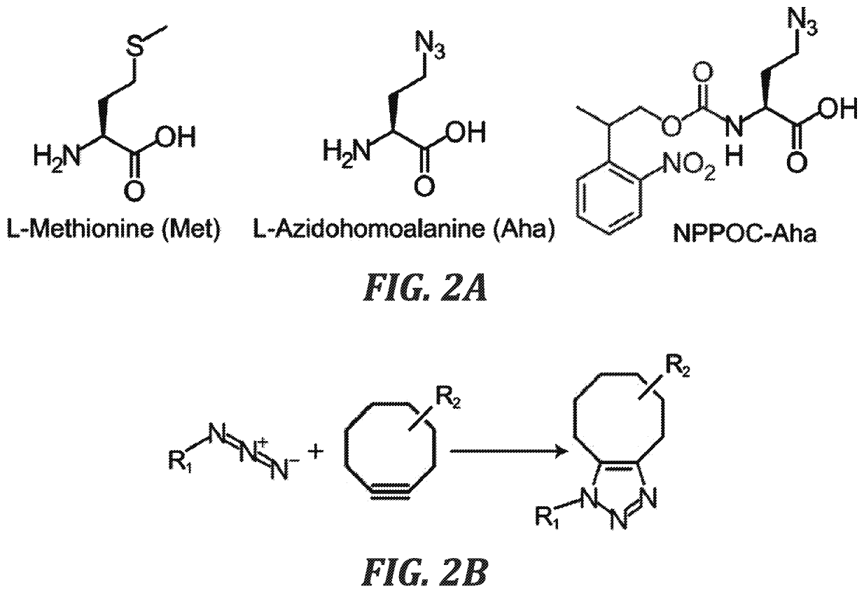

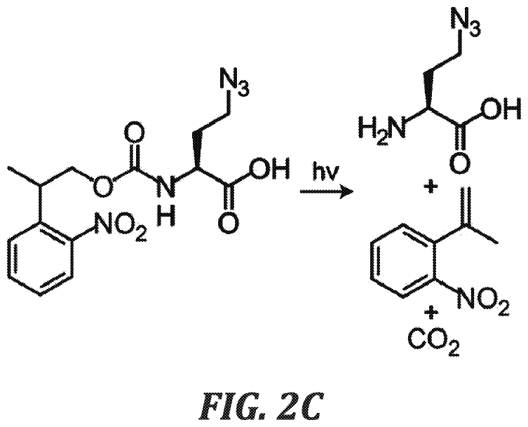

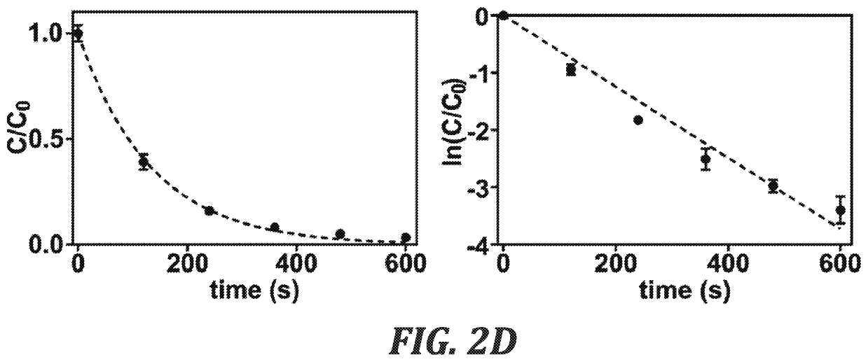

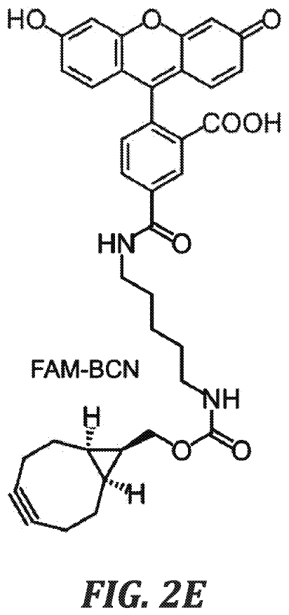

While laBONCAT methodologies can be theoretically applied to any amino acid (including stable isotopes of natural amino acids, ncAAs, and other variants useful for quantitative proteomics), its utility using L-Azidohomoalanine (Aha) is first demonstrated herein. Aha is an azide-bearing ncAA that is metabolically incorporated by endogenous cellular machinery as a methionine (Met) surrogate whose low-level incorporation does not significantly alter protein expression (FIG. 2A). Aha's azido functionality represents a useful bioorthogonal handle for subsequent labeling reactions, including the strain-promoted azide-alkyne cycloaddition (SPAAC) (FIG. 2B). A photocaged Aha (NPPOC-Aha, FIG. 2A) was synthesized through condensation of the α-amine of Aha with the activated ester of 2,5-dioxopyrrolidin-1-yl (2-(2-nitrophenyl)propyl) carbonate (NPPOC). As NPPOC-caged amines undergo irreversible β-elimination upon exposure to near-ultraviolet light (λ=365 nm; FIG. 2C), Aha can be photochemically generated in situ in response to mild and cytocompatible light exposure. Without wishing to be bound by theory, it is believed that this is the first example of an amino acid (canonical or otherwise) that has been photocaged at its N-terminus to prevent translation.

For this system to be effective, the kinetics of NPPOC uncaging should be rapid, such that the liberation of free Aha is not rate limiting compared with the biological processes under study. To determine its photolysis kinetics, NPPOC-Aha (dissolved in H₂O:CH₃CN, 50:50) was irradiated with collimated UV light (λ=365 nm, 10 mW cm⁻², 0-600 s exposure). Degradation products were quantitatively analyzed by HPLC, with elution fractions compositionally identified by mass spectrometry. A first-order decay constant of 0.0075±0.0002 s⁻¹ was observed for NPPOC photolysis (FIG. 2D); 90% of the NPPOC cleaved after 5 minutes of mild irradiation (10 mW cm-2), a timescale suitable for many biological applications.

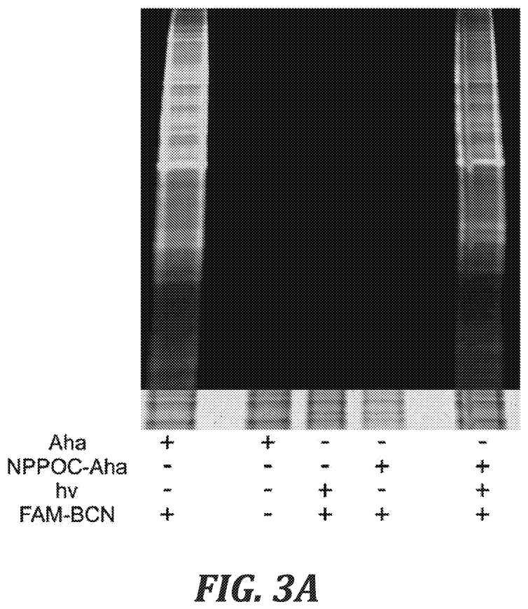

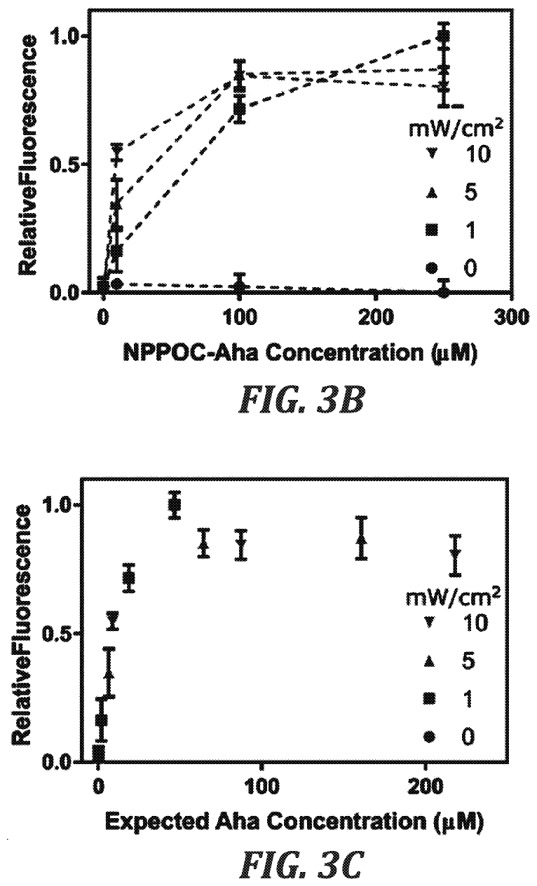

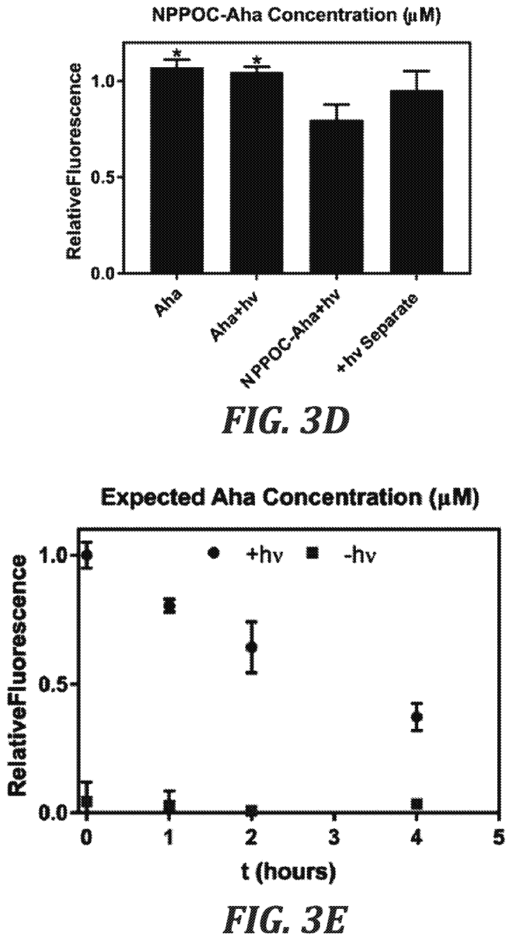

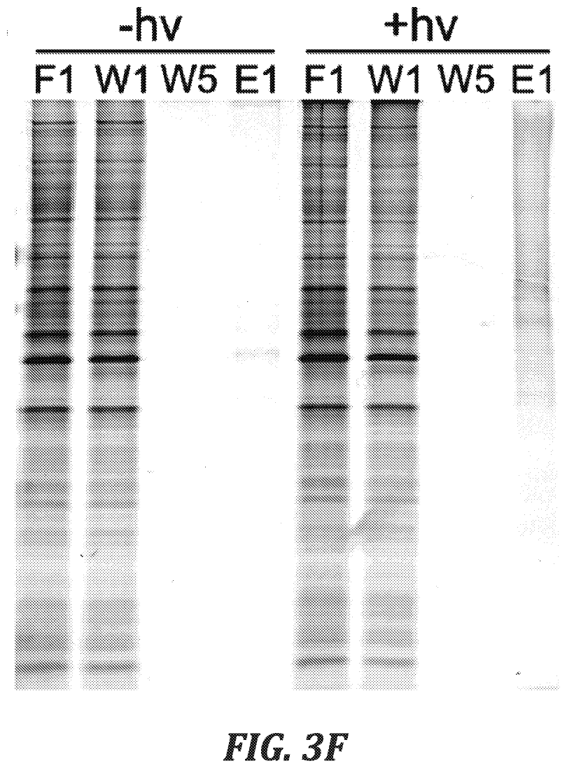

To demonstrate photomediated incorporation of Aha, Met-depleted HeLa cells were incubated with NPPOC-Aha (250 μM). Subsequent irradiation with UV light (λ=365 nm, 10 mW cm⁻², 5 min) yielded photoliberated Aha for metabolic incorporation. Two hours after light exposure, cells were lysed and their proteins were treated with a bicyclononyne-modified fluorescein (FAM-BCN, 100 nM, FIG. 2E) to introduce a fluorescent label by SPAAC. Protein fluorescence was then used to quantify the extent of Aha incorporation following protein separation by sodium dodecyl sulfate polyacrylamide gel electrophoresis (SDS-PAGE). Non-irradiated samples and the Met control exhibited a similar lack of fluorescence; the UV-treated NPPOC-Aha and Aha control displayed significant fluorescent enhancement (FIG. 3A), indicating successful implementation of laBONCAT.

To control the extent of ncAA incorporation into newly synthesized proteins, NPPOC-Aha concentration was varied (0-250 μM) and the intensity of light irradiation (0-10 mW cm⁻²) employed during photo-uncaging. Aha incorporation increased with NPPOC-Aha concentration for a given exposure condition, for low NPPOC-Aha concentrations, metabolic labeling increased with light intensity (FIG. 3B). When the extent of incorporation was normalized for the expected concentration of liberated Aha, based on values predicted by the photokinetics and assuming no side reactions accompanying photolysis, the result was a smooth, continuous curve that plateaus above about 50 μM free Aha (FIG. 3C). To determine the potential effects of UV irradiation on metabolic incorporation, labeled protein fluorescence was compared for samples treated with Aha (50 μM)+/−light (10 mW cm⁻², 5 min) (FIG. 3D). Finding no statistical difference in protein labeling following UV irradiation, incorporation of Aha and irradiated NPPOC-Aha cultures (each at 100 μM) was compared. NPPOC-Aha+light gave rise to slightly less incorporation than Aha alone, which was attributed to incomplete photoconversion of NPPOC-Aha to Aha. This is supported by data that protein labeling does not depend on whether NPPOC-Aha is irradiated separately or during incubation with cells (FIG. 3D).

To assess its in vitro stability, NPPOC-Aha (100 μM) was incubated in media with HeLa cells for 0-4 hr prior to light exposure (10 mW cm⁻², 5 min) and subsequent metabolic labeling (2 hr). Aha incorporation was observed for all irradiated samples, though its extent decreased over time. This was attributed to unknown cellular processing of NPPOC-Aha; simple hydrolysis yielding free Aha did not explain this behavior, as non-irradiated samples did not show increased incorporation over time. While the >4 hours of working time is likely sufficient for many applications, different photocages and/or ncAAs may exhibit increased long-term stability. Such stability is useful in sampling biological systems, as it decouples media swaps from proteome labeling, allowing researchers to standardize their experimental conditions.

After demonstrating the ability to label newly synthesized proteins, the laBONCAT methodologies were extended to their affinity purification. After NPPOC-Aha uncaging and metabolic incorporation of the ncAA, proteins were biotinylated via SPAAC with a dibenzocyclooctyne-modified biotin probe. Biotinylated proteins were captured on a streptavidin resin prior to protein elution by streptavidin denaturation. Eluents were subjected to SDS-PAGE and silver stained for visualization (FIG. 3F). Results highlight the capability to selectively isolate newly synthesized proteins from irradiated samples.

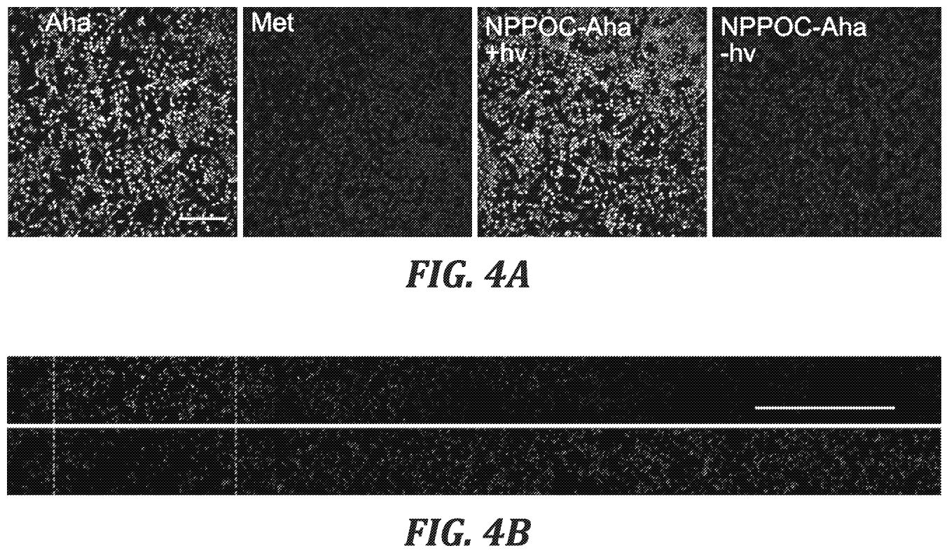

Building on the capability to fluorescently tag cellular lysates as well as isolate species of interest via laBONCAT, the technique's unique ability to label newly synthesized proteins in vitro with spatial control was investigated; this method could be applied to the isolation of proteins transcribed at user-specified times and locations from heterogeneous features in tissue culture, especially tissue slices. HeLa cells were treated with NPPOC-Aha (100 μM) and exposed to light (λ=365 nm, 10 mW cm⁻², 5 min). Two hours after exposure, cells were fixed, permeabilized, and treated with FAM-BCN (100 nM) to introduce a fluorescent label via SPAAC. The extent of fluorescent labeling of cells treated in this method was similar to free Aha, while NPPOC-Aha in the absence of light exhibited low-level labeling similar to Met (FIG. 4).

Next, the ability to control Aha incorporation spatially within synthetic tissues was demonstrated. Cells were encapsulated in oxime-based poly(ethylene glycol) hydrogels (7 wt %), treated with NPPOC-Aha and selectively irradiated through a slitted photomask. Cells were fixed and fluorescently labeled with FAM-BCN, phalloidin, and Hoechst. The observed cellular FAM signal was localized near exposed regions, corresponding to patterned Aha incorporation. FAM fluorescence decreased exponentially away from exposed regions in a diffusion-predicted manner. Actin and DNA staining lack substantial patterning (FIG. 4).

This technique provides an inherent improvement to traditional BONCAT by allowing for precise timing in metabolic incorporation. In addition, the photochemical nature of the system provides a degree of spatial control for the investigation of heterogeneous biological systems. It is believed that laBONCAT can be readily combined with strategies for pulsed stable isotope labeling by amino acids in cell culture (pSILAC) to purify, identify, and quantify proteins expressed at user-defined regions in culture. For example, both photosensitive heavy isotope-labeled amino acid and photosensitive caged non-canonical amino acid can be added to a cell/tissue culture. The amino acids can be irradiated and activated. The cell/tissue culture can be incubated for a period of time, then the cells can be lysed. The proteins of interest can be extracted by coupling to affinity probe (e.g., biotin) and subjected to affinity column chromatography. The heavy isotopes can be compared in MS/MS to compare proteins of interest under different stimuli (from different treatments/cultures). This newfound ability is particularly useful in the investigation of heterogeneous protein-related disease (e.g., Alzheimer's), potentially yielding new diagnostic markers and therapeutic targets.

### Synthesis of (2S)-4-azido-2-({[2-(2-nitrophenyl)propoxy]carbonyl}amino)butanoic Acid (NPPOC-Aha)

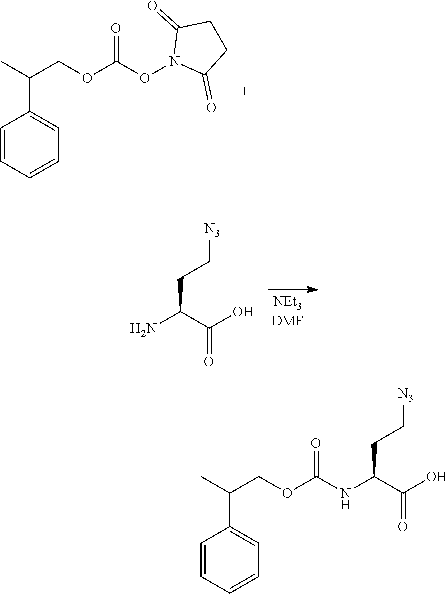 [SMILES: CC(COC(=O)ON1C(=O)CCC1=O)c1ccccc1 ; [N-]=[N+]=NCC[C@H](N)C(=O)O ; CC(COC(=O)N[C@@H](CCN=[N+]=[N-])C(=O)O)c1ccccc1]

L-Azidohomoalanine (Aha) and NPPOC-NHS were synthesized as described previously. Aha-HCl salt (10 mg, 0.055 mmol) was added to a reaction vial. NPPOC-NHS (20.7 mg, 0.64 mmol) was dissolved in dry dimethylformamide (DMF, 1 mL, Acros, AC326870010) and added to the vial containing Aha-HCl. Dry triethylamine (NEt3, 0.05 mL, 0.36 mmol; Sigma, 471283) was added via syringe, and the components stirred overnight at room temperature. The reaction mixture was then concentrated under reduced pressure and dissolved in 30% acetonitrile (CH₃CN) in deionized water (dH₂O). The product was purified by reverse-phase high-performance liquid chromatography (HPLC), eluting with a CH₃CN/dH₂O gradient ramping from 30% to 100% CH3CN over 55 minutes. 20 mg of pure product (denoted NPPOC-Aha) was obtained; quantitative yield. ¹H NMR (500 MHz, CD3OD) δ 7.66 (1H, d, J=8.37 Hz), 7.53 (2H, cm), 7.33 (1H, m, J=4.05 Hz), 4.14 (3H, cm), 3.49 (1H, m, J=6.8 Hz), 3.29 (2H, cm), 1.96 (1H, cm), 1.74 (1H, cm) 1.25 (3H, dd, J=3.3 Hz). HRMS (ESI+) calculated for C₁₄H₁₈N₅O₆ [M+1H]+, 352.1257; observed 352.1259 (Δ=0.6 ppm).

### Synthesis of 5-{[5-({[(1R,8S,9S)-bicyclo[6.1.0]non-4-yn-9-ylmethoxy]carbonyl}amino) pentyl]carbamoyl}-2-(3-hydroxy-6-oxoxanthen-9-yl)benzoic Acid (FAM-BCN)

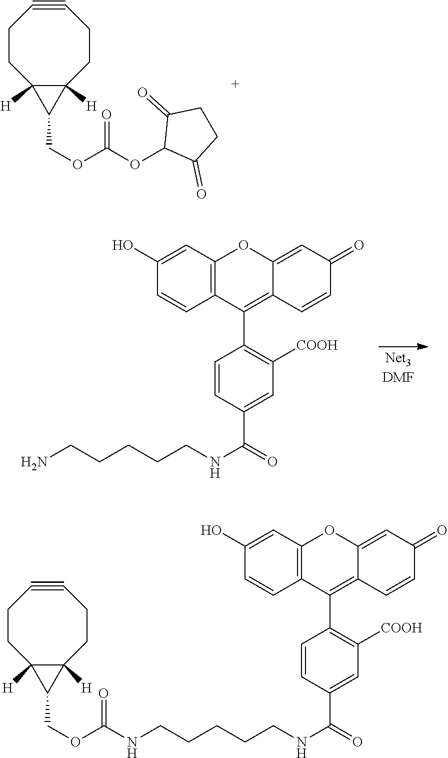 [SMILES: O=C(OC[C@@H]1[C@@H]2CCC#CCC[C@@H]21)OC1C(=O)CCC1=O ; NCCCCCNC(=O)c1ccc(-c2c3ccc(=O)cc-3oc3cc(O)ccc23)c(C(=O)O)c1 ; O=C(NCCCCCNC(=O)c1ccc(-c2c3ccc(=O)cc-3oc3cc(O)ccc23)c(C(=O)O)c1)OC[C@@H]1[C@@H]2CCC#CCC[C@@H]21]

A fluorescent bicyclononyne was necessary for labeling the metabolic incorporation of Aha. (1R,8S,9s)-bicyclo[6.1.0]non-4-yn-9-ylmethyl (2,5-dioxopyrrolidin-1-yl) carbonate (BCN-OSu) was synthesized as described previously. (6)-Carboxyfluorescein cadaverine (20 mg, 0.035 mmol; AAT Bioquest, 127) and BCN-OSu (15 mg, 0.052 mmol) were combined in a reaction vial. The reactants were dissolved in dry DMF (1.5 mL) and dry NEt₃ (0.05 mL, 0.36 mmol) was added via syringe. The solution was a red suspension until the addition of NEt₃. After one hour, the reaction was dark orange and translucent. The reaction was left stirring overnight at room temperature. The reaction was then concentrated under reduced pressure and dissolved in 50:50 dH₂O:CH₃CN. The product was purified by reverse-phase HPLC, eluting with a CH₃CN/dH₂O gradient ramping from 40% to 80% CH₃CN over 55 minutes. 25 mg of pure product (denoted FAM-BCN) was obtained; quantitative yield. HRMS (ESI+) calculated for C₃₇H₃₇N₂O₈ [M+1H]+, 637.2550; observed 637.2556 (Δ=0.9 ppm).

### Synthesis of Benzaldehyde-Functionalized 4-Arm PEG

2,5-dioxopyrrolidin-1-yl 4-formylbenzoate was synthesized as described previously.

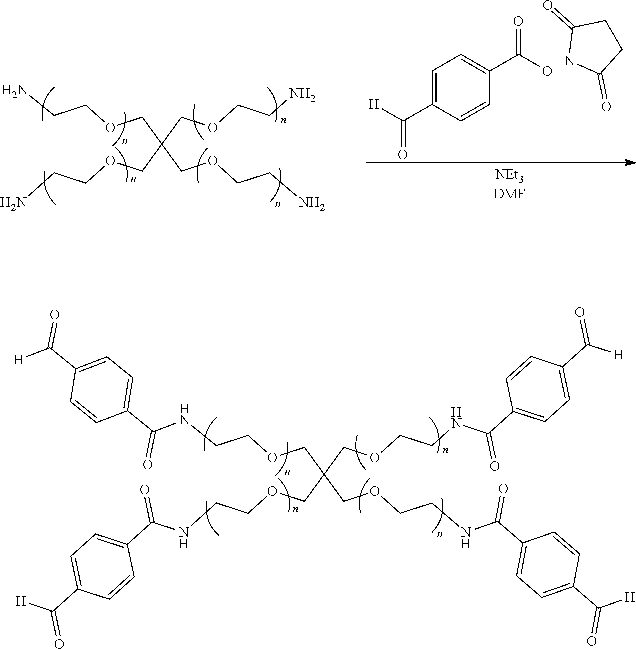

4-arm PEG-amine (524 mg, 0.0524 mmol, Mn˜10 kDa) and 2,5-dioxopyrrolidin-1-yl 4-formylbenzoate (78 mg, 0.32 mmol) were added to a round-bottom flask under Ar. The reactants were dissolved in anhydrous DMF (2.62 mL), N,N-diisopropylethylamine (150 μL, 0.86 mmol) was added, and the reaction was stirred overnight at room temperature. dH₂O (9 mL) was added to the reaction and the solution was dialyzed against dH₂O (Spectra/Por 1 kDa molecular weight cut-off [MWCO]) for 24 h. The retentate was filtered (0.2 μm, polyethersulfone [PES]) and lyophilized to yield a white powder (511 mg, 0.0485 mmol, 92.6% yield). ¹H NMR (500 MHz, CDCl₃) δ 10.1 (s, 2H) 8.14-7.74 (m, 12H), 3.84-3.76 (m, 6H), 3.65 (d, J=21.2 Hz, 1020H [PEG backbone]), 3.56 (t, J=4.7 Hz, 8H), 3.54-3.51 (m, 5H), 3.44 (s, 8H). Based on the relative integrations of protons corresponding to the 4-arm PEG core (δ 3.44) and benzaldehyde (δ 8.14-7.74), end-group functionalization was estimated to be ˜80%.

### Synthesis of Alkoxyamine-Functionalized 4-Arm PEG

2,5-dioxopyrrolidin-1-yl 2-{[(tert-butoxycarbonyl)amino]oxy}acetate was synthesized as described previously.

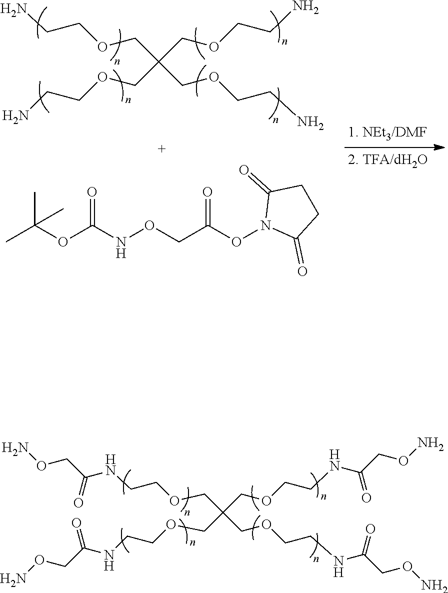

4-arm PEG-amine (110 mg, 0.0110 mmol, Mn˜10 kDa) and 2,5-dioxopyrrolidin-1-yl 2-{[(tert-butoxycarbonyl)amino]oxy}acetate (25.4 mg, 0.0882 mmol) were added to a round-bottom flask under Ar. The reactants were dissolved in anhydrous DMF (1.10 mL), Net (24.5 μL, 0.176 mmol) was added, and the reaction was stirred overnight at room temperature. The reaction was precipitated with diethyl ether (Et₂O, 20 mL), centrifuged, decanted, and re-dissolved in trifluoroacetic acid in dH₂O (5 v/v %, 2 mL). The solution was stirred for 3 h at room temperature. The solution was precipitated with Et₂O (20 mL), centrifuged, decanted, and re-dissolved in dH₂O (9 mL). The solution was dialyzed against dH₂O (Spectra/Por 1 kDa MWCO) for 24 h. The retentate was filtered (0.2 μm, PES) and lyophilized to yield a white powder (59.1 mg, 0.0591 mmol, 53.7% yield). ¹H NMR (500 MHz, CDCl₃) δ 4.57 (s, 1H), 4.53 (s, 1H), 4.36 (s, 1H), 4.26 (s, 1H), 4.21 (s, 1H), 3.99 (s, 2H), 3.60-3.55 (m, 6H), 3.44 (s, 1020H [PEG backbone]), 3.34-3.31 (m, 14H), 3.31-3.28 (m, 9H), 3.21 (s, 8H). Based on the relative integrations of protons corresponding to the 4-arm PEG core (δ 3.44) and the alkoxyamine α-methylene (δ 4.57-3.99), end-group functionalization was estimated to be ˜80%.

### Determination of NPPOC-Aha Photo-Uncaging Kinetics

To determine the kinetics of NPPOC-Aha uncaging upon UV light exposure, NPPOC-Aha (3.4 mM dissolved in 50:50 dH₂O:CH₃CN) was placed in quartz NMR tubes and irradiated with collimated UV light (365 nm, 10 mW/cm²) emanating from an OmniCure (series 1500; light intensity measured with Cole-Palmer series 9811 radiometer). Time points were collected every 120 s for a total of 600 s. The resulting degradation products were separated by reverse-phase HPLC, eluting with a CH₃CN/dH₂O gradient ramping from 5% to 100% CH₃CN over 55 minutes. Remaining NPPOC-Aha was identified by HRMS (ESI+). Calculated for C₁₄H₁₈N₅O₆ [M+1H]+, 352.1257; observed 352.1259 (Δ=0.5 ppm). The HPLC peak corresponding to starting material was integrated and monitored over time.

### Cell Culture, Metabolic Labeling, and Analysis of Aha Incorporation

For in vitro work, borosilicate well plates (Mattek, P35G-1.5-14-C) were pretreated with high glucose Dulbecco's Modified Eagle Media (DMEM) supplemented with fetal bovine serum (FBS, 10%) and penicillin-streptomycin (PS, 1%). Plates were incubated at 37° C. for 10 minutes. HeLa cells were cultured on tissue culture polystyrene before seeding on borosilicate glass. After cells reached the desired confluency (50-60% for imaging; 80-90% for lysate analysis), cells were subjected to the following experiments.

Cells were rinsed with warm 1× Dulbecco's phosphate buffered saline (DPBS) and starved of methionine (Met) by the addition of Met-depleted media (1 h, 37° C.). Met-depleted media was prepared by adding L-cystine-2HCl (0.2 mM), L-glutamine (4 mM), and sodium pyruvate (1 mM) to depleted media (Fisher, 21013024). Media was removed and cells were treated with media containing NPPOC-Aha at the desired concentration (0-250 μM). Control media consisted of Aha or Met in the place of NPPOC-Aha. Cells were then irradiated with light (365 nm, 1-10 mW/cm2, 5 min). The cultures were then incubated (2 h, 37° C.) to promote Aha incorporation. The cells were then washed with 1×DPBS (37° C.).

Samples were prepared for sodium dodecyl sulfate polyacrylamide gel electrophoresis (SDS-PAGE) by lysing cells in lysis buffer (10 mM PO4, pH 7.0, 0.5% sodium dodecyl sulfate). Proteins were collected and immediately placed on ice. Free thiols were capped by the addition of iodoacetamide (10 mM, 30 min, room temperature). Azide-containing proteins were labeled by treatment with FAM-BCN (10 μM, 5 hours, room temperature). Excess FAM-BCN was removed by precipitating proteins with 4× sample volume of cold acetone (−20° C.). Lysates were incubated in acetone (1 h, −20° C.) and then subjected to centrifugation (14,000×g, 10 min). Proteins were redissolved in lysis buffer with gentle heating (40° C., 10 min), precipitated once more, and washed with 4× sample volume acetone before drying (20 min). Proteins were then dissolved in SDS-PAGE sample buffer (50 mM Tris-HC, 2% SDS, 10% glycerol, 12.5% β-mercaptoethanol, 0.025% bromophenol blue). Proteins were electrophoretically separated by a 12% polyacrylamide gel by applying a potential of 105 V. After electrophoresis, gels were scanned on a Typhoon FLA9000 fluorescent gel scanner before being stained with coomassie dye.

### Assessing NPPOC-Aha In Vitro Stability

The stability of NPPOC-Aha was conducted using the above protocols for cell culture and SDS-PAGE with minor modifications. Instead of irradiating samples immediately following addition of NPPOC-Aha media, NPPOC-Aha was incubated in the presence of cells for the desired time (0-4 hours) prior to light exposure. Met (3.33 μM) was added to all media, yielding a 30:1 ratio of NPPOC-Aha:Met5.

### Fluorescent Labeling of Phototagged Cells

To fluorescently label Aha-tagged proteins for visualization, cells were rinsed with warm 1×DPBS (37° C.) before fixing with formaldehyde (4%, 10 min, room temperature). Fixed cells were rinsed with 1×DPBS, and permeabilized with Triton™ X-100 (Sigma, 0.5%, 2 min, room temperature). Cells were then washed (3×, 5 min each) with 1×DPBS. Azides were labeled by strain-promoted azide-alkyne cycloaddition (SPAAC) with FAM-BCN (100 nM, 30 min, room temperature) in the dark. Next, labeled cells were washed (3×, 5 min each) with 1×DPBS prior to imaging on a Leica SP8X laser scanning confocal microscope.

### Spatial Labeling of Phototagged Cells in 3D Tissue Culture

HeLa cells were liberated from the tissue culture plate with trypsin, resuspended in FBS-supplemented DMEM, and pelleted by centrifugation. The cells were then gently resuspended in depleted media containing the photocaged Aha. Hydrogel precursors (10 k PEG functionalized with alkoxyamines or benzaldehyde) were combined (1:1 benzaldehyde:alkoxyamine stoichiometry) with cells prior to deposition on a glass-bottomed well plate (7 wt %, 10 mM aniline, 5 million cells/mL). After the gel had formed (30 min), it was inundated with depleted media containing Aha (100 μM 30 min). Excess media was removed by aspiration and a subsection of the gel was irradiated with light (365 nm, 10 mW/cm2, 5 min) through a slitted photomask. Gels were washed with warm 1×DPBS (37° C., 15 min) before fixing with formaldehyde (4%, 1 hr, room temperature). Fixed cells were rinsed with 1×DPBS, and permeabilized with Triton™ X-100 (Sigma, 0.5%, 1 hr, room temperature). Cells were then washed (3×, 1 hr each) with 1×DPBS. Azides were labeled by SPAAC with FAM-BCN (100 nM, 6 hrs, room temperature) in the dark. Next, cells were labeled with Alexa Fluor 594 phalloidin (5 units/mL, Thermo Fisher), and Hoechst 33342 (5 μg/mL; 1 hr, room temperature, Thermo Fisher) to act as a counterstain by labeling actin and DNA, respectively. The gels were then washed (3×, 1 hr each) with 1×DPBS prior to imaging on a Leica SP8X laser scanning confocal microscope. Intensity analysis was conducted using the Fiji image processing package. An intensity profile of carboxyfluorescein staining over the length of the sample provides the expected patterned response. The result correlated well with the solution to the diffusion in the system (r2=0.60). Counterstains consisting of phalloidin (actin) and Hoechst (DNA) demonstrate no substantial patterning.

The approximate diffusion profile was obtained by solving the second-order partial differential equation (1).

$$\frac{\partial C}{\partial t} = D [ \frac{{\partial }^{2} C}{\partial {x}^{2}} ] + R \\ \begin{matrix}Boundary Conditions & Initial Condition \\ \begin{matrix}C ( x \to ∞ ) = 0 \\ C ( x \to - ∞ ) = 1\end{matrix} & C ( t = 0 ) = [ \begin{matrix}1 ; x \leq 0 \\ 0 ; x > 0\end{matrix}\end{matrix} \qquad ( I )$$

Where C is the concentration at time t(s) and position x(m), D(m2s-1) is the diffusion constant, and R is the consumption rate. The consumption rate was assumed to be negligible, and the diffusion constant of L-isoleucine (7.32×10-10 m²s⁻¹) in aqueous solution was used to approximate the diffusion constant of Aha. The solution to the differential equation assuming infinite sink and infinite source boundary conditions, and the step function initial condition provides equation (2).

$$C ( x , t ) = \frac{1}{2} + \frac{1}{2} Erf ( \frac{x}{\sqrt{4 Dt}} ) \qquad ( 2 )$$

With the origin set to the edge of the photomask, the normalized experimental data was compared to the normalized diffusion profile for t=2 h.

### Affinity Purification of Phototagged Proteins

Aha-tagged proteins were purified through affinity-based enrichment using a modified version of a published protocol. 5 Cells were lysed in 0.5% SDS in 20 mM disodium phosphate. Clarified lysate (500 μL, 0.5 mg/mL total protein content) was heated (90° C., 10 min) prior to reduction (10 mM dithiothreitol, 25 min, 56° C.) and thiol alkylation (iodoacetamide, 14 mM). DNA was digested with Benzonase® (Thermo Scientific) and cleared by centrifugation. Proteins were biotinylated via SPAAC with DBCO-biotin probe (26 μM, Click Chemistry Tools, overnight, 20° C.). Tagged proteins were captured with Streptavidin UltraLink™ Resin (Thermo Scientific), washed with SDS (0.2 mL, 1% in PBS, 6×) followed by urea washing (8 M) and cleavage from resin (70° C., 5 min, 10 mM biotin, 7.5 M guanidine HCl, pH=1.5). Eluent buffer was exchanged to 8 M urea, pH=8, and concentrated on a 30 kDa cut-off centrifugation filter (Millipore). Proteins were separated using 10% polyacrylamide gels and visualized by silver stain.

While illustrative embodiments have been illustrated and described, it will be appreciated that various changes can be made therein without departing from the spirit and scope of the disclosure.

1. A method of analyzing a protein composition in a living cell or organism, comprising:
providing a caged non-canonical amino acid, a caged heavy isotope-labeled amino acid, or a combination thereof, to a living cell or organism, wherein the caged amino acid is configured to be uncaged when exposed to a stimulus;
providing a stimulus to uncage the caged non-canonical amino acid or caged heavy isotope-labeled amino acid;
growing the living cell or organism to incorporate the uncaged amino acid in a growing protein; and
analyzing the living cell or organism for incorporation of the non-canonical amino acid, the heavy isotope-labeled amino acid, or both, into a protein.

2. The method of claim 1, wherein the uncaged non-canonical amino acid or uncaged heavy isotope-labeled amino acid is incorporated into backbone of the growing protein.

3. The method of claim 1, wherein the caged non-canonical amino acid and caged heavy isotope-labeled amino acid are not caged on a side chain of the amino acid.

4. The method of claim 1, wherein the caged non-canonical amino acid and caged heavy isotope-labeled amino acid are each independently caged at the alpha-amino terminus, the carboxylic acid terminus, or both of the alpha-amino and the carboxylic acid termini.

5. The method of claim 1, wherein the stimulus is selected from light having a predetermined wavelength, an enzyme, a small molecule, a nucleic acid, a predetermined temperature, a predetermined pH, ultrasound, a reductant, an oxidant, and a predetermined mechanical force.

6. The method of claim 1, wherein analyzing the living cell or organism comprises:
obtaining a cell population,
lysing the cells,
performing mass spectrometry on proteins obtained from cell lysis, and
identifying the proteins containing the non-canonical amino acid, the heavy isotope-labeled amino acid, or both.

7. The method of claim 1, wherein analyzing the living cell or organism further comprises obtaining the time or the location at which the non-canonical amino acid protein is incorporated into the protein.

8. (canceled)

9. The method of claim 1, wherein analyzing the living cell or organism further comprises obtaining both the time and location at which the protein is synthesized in the living cell or organism.

10. The method of claim 1, further comprising providing a plurality of caged non-canonical amino acids, a plurality of caged heavy isotope-labeled amino acids, or a combination thereof, to a living cell or organism, wherein each caged amino acid is configured to be uncaged when exposed to one or more stimuli.

11. The method of claim 1, wherein the caged amino acid is caged at the carboxylic acid terminus and comprises a structure of —C(O)-(caging group), wherein the caging group is selected from alkoxy optionally substituted with C₁-C₆ alkylcarbonyloxy or substituted with cycloalkylcarbonyloxy, heterocycloalkoxy, arylalkoxy optionally substituted with 1, 2, or 3 substituents independently selected from nitro and alkoxy, and a thioalkyl moiety; or
the caged amino acid comprises a caging group having a heterocycloalkyl moiety, wherein at least 2 ring-forming atoms in the heterocylic moiety are derived from the carboxylic acid terminus of the amino acid; or
the caged amino acid is caged on the amino terminus comprises a structure of —NH-(caging group) or —N=(caging group), wherein the caging group is selected from aryl-CH═, heteroaryl-CH═, aryl, arylalkyl, arylcarbonyloxy optionally substituted with 1, 2, or 3 nitro, and arylalkylcarbonyloxy optionally substituted with 1, 2, or 3 nitro; and
any combination thereof.

12. The method of claim 1, wherein the non-canonical amino acid is selected from L-azidohomoalanine, D-propargylglycine, L-propargylglycine, and any combination thereof.

13. The method of claim 1, wherein the heavy isotope-labeled amino acid is a ¹³C-labeled amino acid or a deuterium-labeled amino acids.

14. The method of claim 1, wherein the caged non-canonical amino acid, the caged heavy isotope-labeled amino acid, or both, further comprise a reactive group different from the caging group, wherein the reactive group is optionally an azide or an alkyne.

15. (canceled)

16. The method of claim 14, wherein the reactive group-containing caged amino acid, when incorporated into a protein, is further reacted with a labeling molecule comprising a complementary reactive group.

17. The method of claim 16, wherein the labeling molecule is selected from a fluorescent molecule, a radioactive molecule, a metal, heavy isotope, and an affinity tag.

18. The method of claim 16, wherein the complementary reactive group is selected from azide, alkyne, a phosphine-activated moiety, and a thiol.

19. The method of claim 1, wherein caged non-canonical amino acid and the caged heavy isotope-containing amino acid are inactive in protein synthesis.

20. A caged non-canonical amino acid or caged heavy isotope-labeled amino acid, comprising a caging group at the alpha-amino terminal group, the carboxylic acid terminal group, or both; wherein the caged amino acid is configured to be uncaged when exposed to a stimulus.

21. The caged amino acid of claim 20, wherein the stimulus is selected from light having a predetermined wavelength, an enzyme, a small molecule, a nucleic acid, a predetermined temperature, a predetermined pH, ultrasound, a reductant, an oxidant, and a predetermined mechanical force.

22. The caged amino acid of claim 20, wherein the caged non-canonical amino acid and caged heavy isotope-labeled amino acid are not caged on a side chain of the amino acid.
